(19) United States
(12) Patent Application Publication
(10) Pub. No.: US 2025/0074845 A1
(43) Pub. Date: Mar. 6, 2025

(54) CATALYST AND METHOD FOR OXIDATIVE DEHYDROGENATION OF LOW-CARBON ALKANES TO LIGHT OLEFINS

(71) Applicant: Braskem America, Inc., Philadelphia, PA (US)

(72) Inventors: Ishant Khurana, Philadelphia, PA (US); Graham Gregorich, Philadelphia, PA (US); Matthew Feeney, Philadelphia, PA (US); Scott Mitchell, Philadelphia, PA (US)

(73) Assignee: Braskem America, Inc., Philadelphia, PA (US)

(21) Appl. No.: 18/826,520

(22) Filed: Sep. 6, 2024

Related U.S. Application Data
(60) Provisional application No. 63/536,914, filed on Sep. 6, 2023.

(51) Int. Cl. C07C 5/333; B01J 23/00; B01J 23/83; B01J 37/04; B01J 37/08

(52) U.S. Cl. CPC C07C 5/333; B01J 23/002; B01J 23/83; B01J 37/04; B01J 37/08; B01J 2523/11; B01J 2523/12; B01J 2523/13; B01J 2523/25; B01J 2523/305; B01J 2523/3706; B01J 2523/842; C07C 2521/02

(57) ABSTRACT

A heterogeneous catalyst composition including a metal and boron catalyst with boron dispersed in a molten matrix, where the molten matrix includes a eutectic mixture of alkali metal or alkaline earth metal salts or hydroxides. A process for preparing a heterogeneous catalyst composition, including combining a mixture of alkali metal or alkaline earth metal salts or hydroxides to form a matrix including a eutectic mixture, adding, to the matrix, a boron precursor and at least one metal catalyst precursor to form a catalyst precursor mixture, and heating the catalyst precursor mixture to a temperature of from 390° C. to 750° C. to form the heterogeneous catalyst composition. A process for catalytic oxidative dehydrogenation of hydrocarbons whereby a hydrocarbon-containing feedstock contacts the heterogeneous catalyst composition to generate olefinic compounds.

### BACKGROUND

Light olefins, ethylene and propylene, are important raw materials for the production of polyolefins such as polyethylene (PE) and polypropylene (PP). A common emerging technology to produce light olefins is oxidative dehydrogenation (ODH) of light alkanes. The emerging processes for the ODH of light alkanes rely on the use of a solid particular catalyst that requires frequent regeneration in both fixed and moving bed reactors. Therefore, the emerging ODH technology suffers from low efficiency and is also costly and energy intensive.

Accordingly, there exists a need for a catalyst with a superior catalyst reactivity, product selectivity and yield, while also enabling the ODH of light alkanes process to operate at a relatively lower temperature, further making it more energy and cost efficient.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a heterogeneous catalyst composition including a metal and boron catalyst with boron dispersed in a molten matrix, where the molten matrix includes a eutectic mixture of alkali metal or alkaline earth metal salts or hydroxides. In some embodiments, the metal and boron catalyst include at least one transition metal compound, rare-earth metal compound, alkaline-earth metal compounds or a mixture thereof. In some embodiments, the eutectic mixture of alkali or alkaline earth metal salts or hydroxides has a melting point of less than about 750° C.

In another aspect, embodiments disclosed herein relate to a process for catalytic oxidative dehydrogenation of hydrocarbons including contacting, in a reactor system, a hydrocarbon-containing feedstock with the heterogeneous catalyst composition to generate olefinic compounds. In some embodiments, the contacting occurs in the presence of an oxygen source, where the oxygen source is a purified O₂ stream, an air stream, or a mixture thereof. The oxygen source may optionally include a diluent selected from the group consisting of nitrogen, argon, and helium. In some embodiments, the hydrocarbon-containing feedstock includes at least one light alkane. In some embodiments, the reactor system includes a single reactor or at least a first reactor and a second reactor connected in a continuous loop for catalyst circulation.

In yet another aspect, embodiments disclosed herein relate to a process for preparing a heterogeneous catalyst composition, including combining a mixture of alkali metal or alkaline earth metal salts or hydroxides to form a matrix including a eutectic mixture, adding, to the matrix, a boron precursor and at least one metal catalyst precursor to form a catalyst precursor mixture, and heating the catalyst precursor mixture to a temperature of from 390° C. to 750° C. to form the heterogeneous catalyst composition. In some embodiments, the process includes preparing the heterogeneous catalyst composition outside of the reactor system and loading the heterogeneous catalyst composition into the reactor system. In some embodiments, the process includes preparing the heterogeneous catalyst composition inside the reactor system.

Other aspects and advantages of the claimed subject matter will be apparent from the following description and the appended claims.

### BRIEF DESCRIPTION OF DRAWINGS

FIG. 1 shows catalyst selectivity and conversion data for Example 1 and Comparative Example 1.

FIG. 2 is a ternary phase diagram for lithium, sodium and potassium carbonates as reproduced from Chen et al. J. Sol. Energy Eng 136(3): 031017 (August 2014).

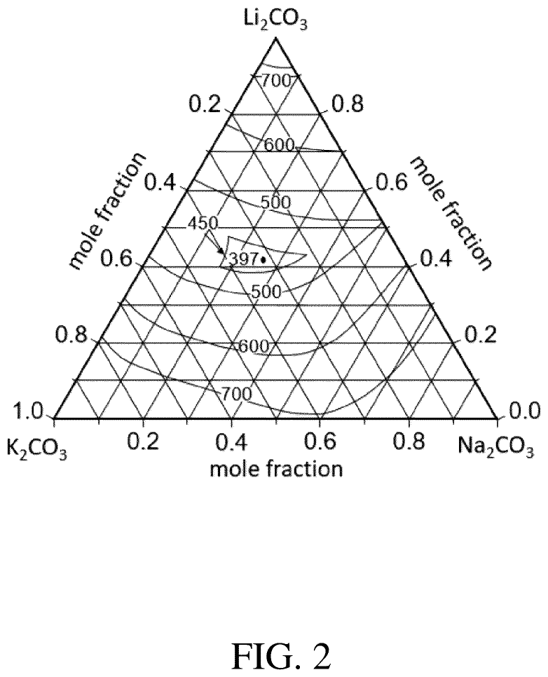

### DETAILED DESCRIPTION

Embodiments disclosed herein relate to a heterogeneous catalyst composition, a process to prepare the heterogeneous catalyst composition, and a process for oxidative dehydrogenation (ODH) of hydrocarbons to form olefinic monomers using the heterogeneous catalyst composition. In particular, embodiments described herein are directed to a heterogeneous catalyst containing a metal and boron catalyst dispersed in a molten matrix. The heterogeneous catalyst composition may be used in a process for ODH of hydrocarbons to form olefinic monomers with improved reactivity, product selectivity and yield by using a chemical looping reactor system, while also enabling the ODH process to operate at a relatively lower temperature compared to commercially available ODH technology. Embodiments described herein also relate a process to prepare the heterogeneous composition either inside or outside a reactor system. The liquid form of the heterogeneous catalyst composition of one or more embodiments may be readily recirculated in the reactor system.

### Heterogeneous Catalyst Composition

One or more embodiments relates to a heterogeneous catalyst composition containing a metal and boron catalyst comprising boron dispersed in a molten matrix, wherein the molten matrix comprises a eutectic mixture of alkali metal or alkaline earth metal salts or hydroxides.

The use of the terms “a” and “an” and “the” and similar referents in the context of describing the elements (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein may be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “dispersion”, “dispersed” and the like refer to a system in which particles of one material are distributed in a continuous phase of another material. The two phases may be in the same or different states of matter, i.e., a solid metal catalyst dispersed in a molten matrix comprising a eutectic mixture. Dispersions are classified in several ways, including how large the dispersed particles are in relation to the particles of the continuous phase, and whether or not a spontaneous precipitation (sedimentation) occurs. In general, dispersions of particles sufficiently large for sedimentation are called suspensions, while those of smaller particles are called colloids, and even smaller are called solutions. In accordance with embodiments of the present disclosure, the metal and boron catalyst dispersed in the molten matrix can be a suspension, or it can be a colloid, or it can be a solution.

As used herein, the term “eutectic” or “eutectic mixture” refers to a homogeneous mixture of substances that melts or solidifies at a single temperature that is lower than the melting point of any of the constituents. It does not necessarily refer to the lowest melting point that is achievable with any particular mixture of substances, this is the eutectic point for those substances, and it may be part of the eutectic mixture. As long as a mixture of substances melts at a temperature lower than the melting point of any of its constituting pure substances, and forms a single continuous phase, it is a “eutectic” or “eutectic mixture” for the purposes of this disclosure. As used herein, the phrase “a eutectic mixture of alkali metal or alkaline earth metal salts or hydroxides” may be alternatively recited as “a eutectic mixture of alkali metal salts, alkali metal hydroxides, alkaline earth metal salts, alkaline earth hydroxides, or a mixture of any two or more thereof”

As used herein, the terms “salt(s)”, “carbonate(s)”, “nitrate(s)”, “acetate(s)” and the like also include their hydrate or solvate forms.

The heterogeneous catalyst composition may contain a metal and boron catalyst. As used herein, the term “metal and boron catalyst” refers to the presence of boron and one or more metals within the dispersed phase of the heterogeneous catalyst, wherein the metal and the boron may be present in the same or in different compounds or alloys. The way boron is incorporated into the dispersed phase of the heterogeneous catalyst is not particularly limited and it may be in the form of boron-containing compounds, boron-containing metal compounds such as boron-containing transition metal compounds and boron-containing rare-earth metal compounds, alloys formed between one or more metals according to the present disclosure and boron, and mixtures thereof.

The metal and boron catalyst may comprise at least one metal compound, which is preferably selected from transition metal compounds, rare-earth metal compounds, alkaline-earth metal compounds or mixtures thereof. In one or more embodiments, the metal and boron catalyst comprises at least one transition metal compound. In one or more embodiments, the metal and boron catalyst comprises at least one rare-earth metal compound. In one or more embodiments, the metal and boron catalyst comprises at least one alkaline-earth metal compound. The metal compound may be a boron-containing compound and/or a boron-absent compound. Transition metal compounds according to the present disclosure may include boron-containing transition metal compounds and/or boron-absent transition metal compounds. Rare-earth metal compounds according to the present disclosure may include boron-containing rare-earth metal compounds and/or boron-absent rare-earth metal compounds. Alkaline-earth metal compounds may include boron-containing alkaline-earth metal compounds and/or boron-absent alkaline-earth metal compounds. The metal of the metal and boron catalyst may also be in the form of alloys formed between one or more metals according to the present disclosure and boron.

Examples of metal compounds may include transition metal compounds, where the transition metal belongs to groups 4-12 in the periodic table of elements, including but not limited to elements V, Cr, Mn, Fe, Co, Ni, Cu, Mo, Zn, W, and mixtures thereof, with the metal being in the oxidized state or in any transition state. More preferably, the transition metal is selected from V, Ni, Cu, Mn, or Fe, or their combinations. “Rare-earth metals” in the context of the present disclosure include lanthanide metals as well as yttrium and scandium. Examples of rare-earth metal compounds include, but are not limited to compounds where the rare-earth metal is selected from elements La, Ce, Pr or Nd. More preferably, the rare-earth metal is selected from La, Ce, or their combination. Alkaline-earth compounds are those where the metal belongs to group 2 of the periodic table of elements and are preferably selected from Ba.

In one or more embodiments, the metal of the metal and boron catalyst is in the oxidized form (i.e., one or more of its cationic forms), and it is dispersed in the molten matrix. For the purposes of the present disclosure, the metal catalyst may be in the oxidized state or in any transition state. It is envisioned that the metal compounds may be boron-containing compounds or boron-absent compounds. Preferably, the metal compounds may take the form of oxides or salts, such as transition metal carbonates, transition metal salts of an organic acid, transition metal oxides, rare-earth metal carbonates, rare-earth metal salts of an organic acid, rare-earth metal oxides, alkaline-earth metal carbonates, alkaline-earth metal salts of an organic acid, alkaline-earth metal oxides, with the metal being in any transition state.

In one or more embodiments, the metal and boron catalyst comprise at least two metals and boron, such as two, three or four metals and boron. The at least two metals are preferably selected from transition metals, rare-earth metals, alkaline-earth metals, and mixtures thereof. The at least two metals may be at least two transition metals, at least two rare-earth metals, at least two alkaline-earth metals or a mixture of two or more metals selected from transition metals, rare-earth metals, and alkaline-earth metals. More preferably, the metals of the metal and boron catalyst comprise a combination of transition metal and rare-earth metal, or a combination of transition metal, rare-earth metal, and alkaline-earth metal. In one or more embodiments, the metal and boron catalyst may include B, La and Fe. In one or more embodiments, the metal and boron catalyst may include B, La, Fe and Ba. In one or more embodiments, the metal and boron catalyst may include B, Cu and Ce. In one or more embodiments, the metal and boron catalyst may include B, Cu, Ce and Ba.

The loading of boron within the metal and boron catalyst (i.e., dispersed phase) may be represented as a molar ratio between the elemental boron within the dispersed phase and the molten matrix (B:Eu). In one or more embodiments of the present disclosure, the loading of boron may be from about 0.5:1 to about 50:1, represented as a molar ratio between the elemental boron within the dispersed phase and the molten matrix. For example, the loading of boron may be in a range having a lower limit of about 0.5:1, 1:1 1.5:1 or 1.8:1, and an upper limit of about 50:1, 45:1, 40.5:1, 30:1, 20:1, 10:1, 5:1 represented as a molar ratio between the elemental boron within the dispersed phase and the molten matrix.

The term “about” in this disclosure will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

In one or more embodiments, the metal and boron catalyst includes boron, transition metal (TM), rare-earth metal (REM) and alkaline-earth metal (AEM) in a mol % ratio B:TM:REM:AEM of about 3-5: 30-40:1-2:1. In a preferred embodiment, the metal and boron catalyst includes B, Fe, La and Ba in a mol % ratio B:Fe:La:Ba of about 3-5: 30-40:1-2:1.

In one or more embodiments, the metal and boron catalyst includes boron, transition metal (TM), rare-earth metal (REM) and alkaline-earth metal (AEM) in a mol % ratio to molten matrix (Eu) B:TM:REM:AEM:Eu of about 3-5: 30-40:1-2:1: 4-5. In a preferred embodiment, the metal and boron catalyst includes B, Fe, La and Ba in a mol % ratio to molten matrix B:Fe:La:Ba:Eu of about 3-5: 30-40:1-2:1: 4-5.

In particular embodiments, such metals may be dispersed in a molten matrix, where the molten matrix includes a eutectic mixture of alkali metal or alkaline earth metal salts or hydroxides. The eutectic mixture of alkali metal or alkaline earth metal salts or hydroxides may be a binary or ternary mixture.

In one or more embodiments, the eutectic mixture of alkali metal or alkaline earth metal salts or hydroxides may have a melting point of less than about 750° C., such as less than about 650° C., less than about 600° C., less than about 500° C. or less than about 400° C. In one or more embodiments, the eutectic mixture of alkali metal or alkaline earth metal salts or hydroxides may have a melting point in a range of from about 390° C. to about 750° C. For example, the melting point may be in a range having a lower limit of about 390° C., 450° C., and 550° C., and an upper limit of about 600° C., 650° C., and 750° C., where any lower limit and upper limit may be used in combination.

In one or more embodiments, the salts of the eutectic mixture of alkali metal or alkaline earth metal salts or hydroxides may be selected from carbonates, chlorides, nitrates, nitrites, or mixtures thereof.

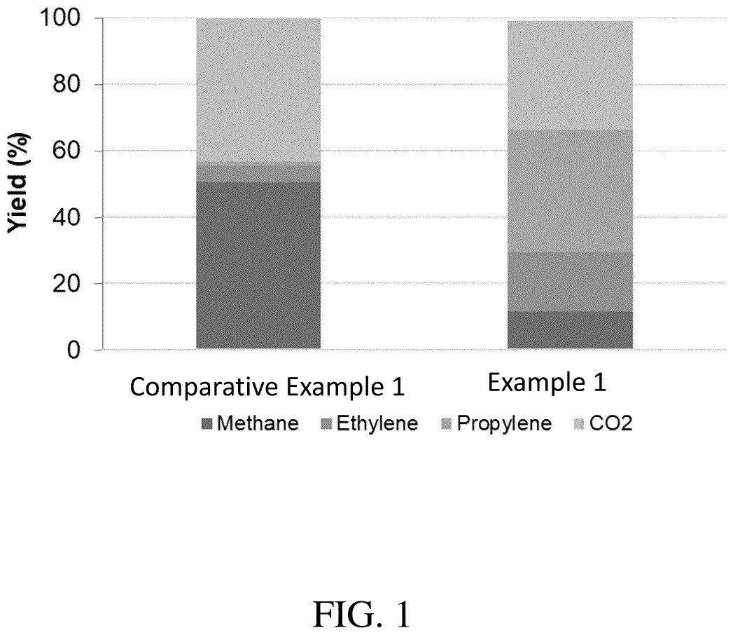

In one or more embodiments, the eutectic mixture of alkali metal or alkaline earth metal salts or hydroxides may be a binary or ternary salt or hydroxide mixture, more preferably a binary or ternary salt mixture. In an even more preferred embodiment, the binary or ternary salt mixture are mixtures of carbonates, more preferably carbonates selected from Li, Na, and K carbonates. In some embodiments, the eutectic mixture comprises two or more of Li₂CO₃, Na₂CO₃, and K₂CO₃. FIG. 1 is a phase diagram reproduced from Chunlin Chen, Ty Tran, Rene Olivares, Steven Wright, Shouyi Sun; J. Sol. Energy Eng. August 2014, 136(3): 031017, illustrating the melting points for a wide variety of Li, Na, and K carbonates mixtures. Other illustrative eutectic mixture of alkali metal carbonates includes those in Table 1, reproduced from Mutch et al. J. Mater. Chem. A 7, 12951-12973 (2019).

TABLE 1

| | Melting Point |
|---|---|
| Salt System | (° C.) |
| Li₂CO₃ | 723 |
| Na₂CO₃ | 854 |
| K₂CO₃ | 891 |
| Li₂CO₃—Na₂CO₃ (52-48 mol %) | 501 |
| Li₂CO₃—K₂CO₃ (62-38 mol %) | 498 |
| Na₂CO₃—K₂CO₃ (56-44 mol %) | 710 |
| Li₂CO₃—Na₂CO₃—K₂CO₃ (43.5-31.5-25 mol %) | 397 |
| Na₂CO₃—BaCO₃ (52.2-47.3 mol %) | 686 |

For the purposes of the present disclosure, the proportions, either molar or by weight, between the alkali metal or alkaline earth metal salts or hydroxides within the molten matrix is not particularly limited as long as the resulting mixture melts or solidifies at a single temperature that is lower than the melting point of any of the constituents. In other words, the proportions between the constituents of the molten matrix are those enough to result in a eutectic mixture.

In particular embodiments, the heterogeneous catalyst composition may contain a molten matrix containing a eutectic mixture of carbonates of Li and Na in a mole ratio of about 52:48, respectively, or a eutectic mixture of carbonates of Li and K in a mole ratio of about 62:38, respectively, or a eutectic mixture of carbonates of Li, Na, and K, in a mole ratio of about 43:32:25, respectively. More preferably, the heterogeneous catalyst composition may contain a molten matrix containing a eutectic mixture of carbonates of Li, Na, and K, in a mole ratio of about 43:32:25, respectively.

In one or more embodiments, the metal and boron catalyst may include B, La, Fe, and Ba, and the molten matrix may be a ternary mixture of carbonates of Li, Na, and K. In one or more embodiments, the metal and boron catalyst may include B, Cu, Ce and Ba, and the molten matrix may be a ternary mixture of carbonates of Li, Na and K.

### Preparation of Heterogeneous Catalyst Composition

One or more embodiments disclosed herein relate to a process to prepare a heterogeneous catalyst composition. Specifically, one or more embodiments relates to preparation of a heterogeneous catalyst composition by combining a mixture of alkali metal or alkaline earth metal salts or hydroxides to form a matrix comprising a eutectic mixture and adding to the matrix a boron precursor and at least one metal catalyst precursor to form a catalyst precursor mixture. Preparation of the heterogeneous catalyst composition may also include heating the catalyst precursor mixture. The heterogeneous catalyst composition may be prepared either inside or outside of a reactor system.

In one or more embodiments, the heterogeneous catalyst composition is prepared inside a reactor system. The heterogeneous catalyst composition may be recirculated readily in the reactor system, due to the liquid form of the heterogeneous catalyst composition of one or more embodiments. The reactor system may include a tubular reactor, a continuous stirred tank reactor (CSTR), or a loop reactor.

In one or more embodiments, the reactor system may be operated as a continuous process, a semi-continuous process, or a batch process.

In one or more embodiments, the reactor system includes a single reactor. In some embodiments, the reactor system includes at least a first reactor, and a second reactor connected in a continuous loop for catalyst circulation.

In one or more embodiments, a heterogeneous catalyst composition is prepared by combining a eutectic mixture of alkali metal or alkaline earth metal salts or hydroxides to form a molten matrix, a boron precursor and at least one metal catalyst precursor to form a catalyst precursor mixture.

In one or more embodiments, the catalyst precursor mixture includes at least one metal catalyst precursor selected from the group consisting of transition metal precursors, rare-earth metal precursors, alkaline-earth metal precursors or a mixture thereof. In one or more embodiments the metal catalyst precursor includes a mixture of transition metal precursor and rare-earth metal precursor. In one or more embodiments the metal catalyst precursor includes a mixture of transition metal precursor, rare-earth metal precursor and alkaline-earth metal precursor.

It is envisioned that the metal catalyst precursor mixture may comprise the at least one metal to be incorporated into the catalyst (i.e., into the boron and metal catalyst, or dispersed phase) in the form of a metal compound such as transition metal compounds, rare-earth metal compounds, alkaline-earth metal compounds or mixtures thereof, or alloys formed between one or more metals according to the present invention and boron. In one or more embodiments, the at least one metal is incorporated in the dispersed phase as the oxidized form (i.e., one or more of its cationic forms), or any transition state.

Preferably, the metal catalyst precursors may be in the form of oxides, hydroxides or salts such as carbonates, nitrates, acetates and metal salts of an organic acid, or mixtures thereof, with the metal being in the oxidized form or any transition state. In particular embodiments, transition metal catalyst precursors according to the present invention may be selected from transition metal oxides, transition metal hydroxides or transition metal salts such as transition metal carbonates, transition metal nitrates, transition metal acetates and transition metal salts of an organic acid, or mixtures thereof, with the metal being in the oxidized form or any transition state. In particular embodiments, rare-earth metal catalyst precursors according to the present invention may be selected from rare-earth metal oxides, rare-earth metal hydroxides or rare-earth metal salts such as rare-earth metal carbonates, rare-earth metal nitrates, rare-earth metal acetates and rare-earth metal salts of an organic acid, or mixtures thereof, with the metal being in the oxidized form or any transition state. In particular embodiments, alkaline-earth metal catalyst precursors according to the present invention may be selected from alkaline-earth metal oxides, alkaline-earth metal hydroxides or alkaline-earth metal salts such as alkaline-earth metal carbonates, alkaline-earth metal nitrates, alkaline-earth metal acetates and alkaline-earth metal salts of an organic acid, or mixtures thereof, with the metal being in the oxidized form or any transition state.

It is envisioned that the metal catalyst precursors may also be boron precursors if boron-containing metal catalyst precursors are used. Hence, for the purposes of the present invention, the term “boron precursor and at least one metal catalyst precursor” may include one or more catalyst precursors containing metal and boron, simultaneously.

Examples of metal catalyst precursors may include transition metal precursors, where the transition metal belongs to groups 4-12 in the periodic table of elements, including but not limited to elements V, Cr, Mn, Fe, Co, Ni, Cu, Mo, Zn, W, and mixtures thereof, with the metal being in the oxidized state or in any transition state. More preferably, the transition metal is selected from V, Ni, Cu, Mn, or Fe, or their combinations. Examples of rare-earth metal precursors include, but are not limited to compounds where the rare-earth metal is selected from elements La, Ce, Pr or Nd, with the metal being in the oxidized state or in any transition state. More preferably, the rare-earth metal is selected from La, Ce or their combination. Alkaline-earth precursors are those where the metal belongs to group 2 of the periodic table of elements, with the metal being in the oxidized state or in any transition state, and are preferably selected from Ba.

In one or more embodiments, the at least one metal catalyst precursor comprise at least two metals, such as two, three or four metals, in a single or in separate precursors. The at least two metals are preferably selected from transition metals, rare-earth metals, alkaline-earth metals, and mixtures thereof. The at least two metals may be at least two transition metals, at least two rare-earth metals, at least two alkaline-earth metals or a mixture of two or more metals selected from transition metals, rare-earth metals, and alkaline-earth metals. More preferably, the metals of the catalyst precursor mixture comprise a combination of transition metal and rare-earth metal, or a combination of transition metal, rare-earth metal, and alkaline-earth metal.

In one or more embodiments, the at least one metal catalyst precursor comprises a transition metal carbonate, transition metal acetate or transition metal nitrate of one or more of V, Cr, Mn, Fe, Co, Ni, Cu, Mo, Zn and W, preferably a transition metal carbonate, transition metal acetate or transition metal nitrate of one or more of V, Ni, Cu, Mn, or Fe.

In one or more embodiments, the at least one metal catalyst precursor comprises a rare-earth metal hydroxide, rare-earth metal acetate or rare-earth carbonate of one or more of La, Ce, Pr, and Nd, preferably a rare-earth metal hydroxide, rare-earth metal acetate or rare-earth carbonate of one or more of La or Ce.

In one or more embodiments, the at least one metal catalyst precursor comprises a rare-earth metal hydroxide, rare-earth metal acetate or rare-earth carbonate of one or more of La, Ce, Pr, and Nd, preferably rare-earth metal hydroxide, rare-earth metal acetate or rare-earth carbonate of one or more of La or Ce.

In one or more embodiments, the at least one metal catalyst precursor may include Fe and La. In more particular embodiments, the at least one metal catalyst precursor includes a mixture of iron carbonate and lanthanum carbonate. In one or more embodiments, the at least one metal catalyst precursor may include Fe, La, and Ba. In more particular embodiments, the at least one metal catalyst precursor includes a mixture of iron carbonate, lanthanum carbonate and barium carbonate. In one or more embodiments, the at least one metal catalyst precursor includes Cu and Ce. In more particular embodiments, the at least one metal catalyst precursor includes a mixture of copper acetate and cerium acetate. In one or more embodiments, the at least one metal catalyst precursor may include Cu, Ce and Ba. In more particular embodiments, the at least one metal catalyst precursor includes a mixture of copper acetate, cerium acetate and barium acetate.

In one or more embodiments, the boron precursor may be selected from boron-containing compounds such as boron oxides, boron salts and boric acid. Boron oxides according to the present disclosure may include boron monoxide and boron trioxide. Boron salts according to the present disclosure may include borax (sodium tetraborate decahydrate), sodium borate, potassium borate, calcium borate, magnesium borate and lithium borate.

In one or more embodiments, the catalyst precursor includes boron, transition metal (TM), rare-earth metal (REM) and alkaline-earth metal (AEM) in a mol % ratio B:TM:REM:AEM of about 3-5: 30-40:1-2:1. In a preferred embodiment, the catalyst precursor includes B, Fe, La and Ba in a mol % ratio B:Fe:La:Ba of about 3-5: 30-40:1-2:1.

In one or more embodiments, the catalyst precursor includes boron, transition metal (TM), rare-earth metal (REM) and alkaline-earth metal (AEM) in a mol % ratio to molten matrix (Eu) B:TM:REM:AEM:Eu of about 3-5: 30-40:1-2:1: 4-5. In a preferred embodiment, the catalyst precursor includes B, Fe, La and Ba in a mol % ratio to molten matrix B:Fe:La:Ba:Eu of about 3-5: 30-40:1-2:1: 4-5.

The molten matrix containing a eutectic mixture of alkali metal or alkaline earth metal salts or hydroxides included in the process may include any of the eutectic mixtures as previously described.

In one or more embodiments, the eutectic mixture of alkali metal or alkaline earth metal salts or hydroxides may be a binary or ternary salt or hydroxide mixture, more preferably a binary or ternary salt mixture. In an even more preferred embodiment, the binary or ternary salt mixture are mixtures of carbonates, more preferably carbonates selected from Li, Na, and K carbonates. In some embodiments, the eutectic mixture comprises two or more of Li₂CO₃, Na₂CO₃, and K₂CO₃.

For the purposes of the present disclosure, the proportions, either molar or by weight, between the alkali metal or alkaline earth metal salts or hydroxides added to the process for preparation of the molten matrix is not particularly limited as long as the resulting mixture melts or solidifies at a single temperature that is lower than the melting point of any of the constituents. In other words, the proportions between the constituents of the molten matrix are those enough to result in a eutectic mixture.

In particular embodiments, the molten matrix containing a eutectic mixture of carbonates of Li and Na in a mole ratio of about 52:48, respectively, or a eutectic mixture of carbonates of Li and K in a mole ratio of about 62:38, respectively, or a eutectic mixture of carbonates of Li, Na, and K, in a mole ratio of about 43:32:25, respectively. More preferably, the molten matrix contains a eutectic mixture of carbonates of Li, Na, and K, in a mole ratio of about 43:32:25, respectively.

The process to prepare the heterogeneous catalyst composition may be carried out either outside or inside of the reactor system. In one or more embodiments, preparing the heterogeneous catalyst composition outside or inside the reactor system includes combining a mixture of alkali metal or alkaline earth metal salts or hydroxides to form a matrix comprising a eutectic mixture, adding to the matrix at least one metal catalyst precursor to form a catalyst precursor mixture, and heating the catalyst precursor mixture to a temperature of about 390° C. to about 750° C. to form the at least one heterogeneous catalyst composition. In one or more embodiments, the heating may occur at a temperature of about 350° C. to about 450° C.

### Process for Oxidative Dehydrogenation of Hydrocarbons

One or more embodiments disclosed herein relate to a process for ODH of hydrocarbons to form olefinic compounds (also referred to as monomers) using at least one heterogeneous catalyst composition as defined in the present disclosure. The process may include contacting, in a reactor system, a hydrocarbon-containing feedstock with the at least one heterogenous catalyst composition according to the present disclosure to generate olefinic compounds.

The reactor system used in the process for ODH of hydrocarbons to form olefinic compounds or olefinic monomers may include any of the reactor systems as previously described.

As described above, the reactor system may include a tubular reactor, a continuous stirred tank reactor (CSTR), or a loop reactor and the reactor system may be operated as a continuous process, a semi-continuous process, or a batch process. The reactor system may include at least a first reactor and a second reactor, where the first and second reactors are connected in a continuous loop for catalyst circulation.

In some embodiments, the reactor system includes a single reactor. In embodiments where the process for ODH of hydrocarbons to form olefinic compounds or olefinic monomers is carried out in a single reactor, the at least one heterogeneous catalyst composition is contacted sequentially: first with a hydrocarbon-containing feedstock, then with an oxygen source.

The process for ODH of hydrocarbons to form olefinic compounds may include contacting at least one heterogeneous catalyst composition with a hydrogen-containing feedstock. The hydrocarbon-containing feedstock may include a refinery range hydrocarbon. In one or more embodiments, the refinery range hydrocarbon includes at least one light alkane, for example ethane, propane, n-butane, isobutene, n-pentane, isopentane, n-hexane, isohexane, and combinations thereof.

In some embodiments, the hydrogen-containing feedstock optionally contains a diluent. The diluent may include nitrogen (N₂), argon (Ar), or helium (He).

In one or more embodiments, the at least one heterogeneous catalyst composition is prepared outside of the reactor system. In this case, the process for catalytic oxidative dehydrogenation of hydrocarbons may include preparing the at least one heterogeneous catalyst composition outside of the reactor system and loading the at least one heterogeneous catalyst composition into the reactor system.

In one or more embodiments, the at least one heterogeneous catalyst composition is prepared inside of the reactor system. In this case, the process for catalytic oxidative dehydrogenation of hydrocarbons may include preparing the at least one heterogeneous catalyst composition inside the reactor system and heating at a temperature of about 390° C. to about 750° C. In one or more embodiments, the heating may occur at a temperature of about 350° C. to about 450° C.

The process for ODH of hydrocarbons to form olefinic compounds may include contacting at least one heterogeneous catalyst composition with an oxygen source. In one or more embodiments, the oxygen source includes a purified oxygen (O₂) stream, an air stream, or a mixture thereof. In some embodiments, the oxygen source optionally contains a diluent. The diluent may include carbon dioxide (CO₂), nitrogen (N₂), argon (Ar), or helium (He).

In one or more embodiments, the process for ODH of a hydrocarbon feedstock to form olefinic compounds using at least one heterogeneous catalyst composition is an exothermic process. In one or more embodiments, the contacting in a reactor system is conducted at a temperature of about 750° C. or less and/or a pressure of about 20 atm Cor less. For example, the contacting process temperature may be in a range having a lower limit of about 0, 250, 500, and 750° C. and an upper limit of about 740, 745, and 750° C., where any lower limit and upper limit may be used in combination. Additionally, for example, the contacting process pressure may be in a range having a lower limit of about 0, 5, 10, 15 and 20 atm and an upper limit of about 19, 19.5, and 20 atm, where any lower limit and upper limit may be used in combination.

The olefinic compound produced by the process for ODH of hydrocarbons described herein may include light olefins, α-olefins, and terminal dienes. Some examples of olefinic compounds may include ethene, propene, 1-butene, 2-methyl-but-1-ene, 1-n-pentene, 1-n-hexene, 2-methyl-pent-1-ene, 3-methyl-pent-1-ene, 1,3-butadiene, 1,3-pentadiene, 1,4-pentadiene, 1,3-hexadiene, 1,4-hexadiene, or 1,5-hexadiene.

### EXAMPLE

The following example is provided for the purpose of further illustrating embodiments described herein and is in no way to be taken as limiting.

### Example 1

Example 1 was prepared by adding 127.2 g of Li₂CO₃, 133.6 g of Na₂CO₃, and 130.0 g K₂CO₃ in a mixing vessel and mixing thoroughly. Upon mixing, the mixture is compositionally similar to a eutectic mixture of the materials in the phase diagram that melts at about 397° C., and may be described by the following chemical formula: Li_{0.862}Na_{0.63}K_{0.50}CO₃. The melting temperature was confirmed by differential scanning calorimetry (DSC) analysis.

Next, a catalyst precursor mixture was prepared by combining 0.200 g of the eutectic mixture of Li_{0.862}Na_{0.63}K_{0.50}CO₃, 3.000 g of FeCO₃, 0.200 g of BaCO₃, 0.200 g of La(OH)₃ and 0.200 g of B₂O₃ in a 20 mL borosilicate glass vial. The total weight of catalyst precursor mixture in the vial was about 4.000 g. After the loading of a vial is completed, the vial is covered with a lid, and the content is mixed by handshaking for 5 min.

After mixing the catalyst precursor mixture, the lid of the glass vial was removed and the glass vial containing the catalyst precursor mixture was placed in a heating metal block, which was pre-heated to 395° C. The open vial was heated in the metal block for 30 minutes to convert the metal precursor compounds to the metal catalyst state as the vial content is heated up and maintained at the 395° C. temperature.

During the heating step, gas has evolved from the catalyst precursor mixture powder particles which is observed visually from motion of powder particles via fluidization and gas channel formation. In addition, a visual color change from a light brown for the metal precursor mixture to a black color indicates that the final catalyst state has been obtained. The off-gas evolving during the heating step is believed to be primarily steam from the crystal hydride dehydration and CO₂ from the transition of rare-earth metal carbonate precursors conversion to a metal oxide form in the final catalyst state. After the heating step, the catalyst is removed from the vial and ground by hand using a mortar and pestle. The final product powder is stored in a sealed glass vial until further use.

### Comparative Example 1

Comparative Example 1 was prepared by the same methods as Example 1, except the catalyst precursor mixture was prepared by combining 0.200 g of the eutectic mixture of Li_{0.862}Na_{0.63}K_{0.50}CO₃, 3.000 g of FeCO₃, 0.200 g of BaCO₃, and 0.200 g of La(OH)₃ in a 20 mL borosilicate glass vial.

Selective Production of Propylene from Propane

### Production Example 1

Production example 1 was a catalyst containing about 60-80 wt % of metal oxide catalyst of Example 1.

### Comparative Production Example 1

Comparative production example 1 was a catalyst containing about 60-80 wt % of a metal-free boron oxide.

A 20 mL micro-batch reactor was loaded with 1.5 mL of propane feed, 0.75 mL of air and nitrogen in the head space, and 500 mg of the catalysts as described in Example 1 for Production example 1 and Comparative example 1 for Comparative production example 1.

For both Production example 1 and Comparative production example 1, the micro-batch reactor was maintained at a temperature of 475° C. for 15 minutes. Selective conversion of olefins is measured using gas chromatography headspace analysis.

Results are shown in FIG. 1, which illustrates that the molten catalyst composition of Example 1 selectively converts olefins more efficiently than the metal-free boron oxide catalyst composition of Comparative Production Example 1.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures.

What is claimed:

1. A heterogeneous catalyst composition comprising a metal and boron catalyst comprising boron dispersed in a molten matrix, wherein the molten matrix comprises a eutectic mixture of alkali metal or alkaline earth metal salts or hydroxides.

2. The heterogeneous catalyst composition of claim 1, wherein the metal and boron catalyst comprises at least one metal compound selected from transition metal compounds, rare-earth metal compounds, alkaline-earth metal compounds or a mixture thereof.

3. The heterogeneous catalyst composition of claim 2, wherein the at least one metal compound is an alloy comprising one or more transition metals, earth metals, alkaline-earth metals, and boron.

4. The heterogeneous catalyst composition of claim 1, wherein the eutectic mixture of alkali metal or alkaline earth metal salts or hydroxides has a melting point of less than about 750° C.

5. The heterogeneous catalyst composition of claim 1 wherein the eutectic mixture of alkali metal or alkaline earth metal salts or hydroxides is a binary or ternary salt or hydroxide mixture.

6. The heterogeneous catalyst composition of claim 5, wherein the eutectic mixture of alkali metal or alkaline earth metal salts or hydroxides is a ternary salt mixture comprising carbonates of Li, Na, and K.

7. The heterogeneous catalyst composition of claim 1, wherein the metal and boron catalyst comprises at least two metals selected from the group consisting of transition metals, rare-earth metals, alkaline-earth metals, and mixtures thereof.

8. The heterogeneous catalyst composition of claim 1 wherein the metal and boron catalyst comprises an elemental boron to molten matrix mole ratio ranging from about 0.5:1 to about 50:1.

9. A process for catalytic oxidative dehydrogenation of hydrocarbons, the process comprising:
contacting, in a reactor system, a hydrocarbon-containing feedstock with the heterogeneous catalyst composition of claim 1 to generate olefinic compounds.

10. The process of claim 9, wherein contacting in the reactor system is conducted at a temperature of about 750° C. or less.

11. The process of any one of claim 9, wherein the process is carried out at a pressure of about 20 atm or less.

12. The process of claim 9, wherein the hydrocarbon-containing feedstock comprises refinery range hydrocarbon.

13. The process of claim 9, wherein the contacting is in the presence of:
an oxygen source, wherein the oxygen source comprises a purified O₂ stream, an air stream, or a mixture thereof; and
optionally, a diluent selected from the group consisting of nitrogen, argon, and helium.

14. The process of claim 9, wherein the reactor system comprises a single reactor or at least a first reactor and a second reactor connected in a continuous loop for catalyst circulation.

15. A process for preparing a heterogeneous catalyst composition of claim 1, the process comprising:
combining a mixture of alkali metal or alkaline earth metal salts or hydroxides to form a matrix comprising a eutectic mixture;
adding, to the matrix, a boron precursor and at least one metal catalyst precursor to form a catalyst precursor mixture; and
heating the catalyst precursor mixture to a temperature of from 390° C. to 750° C. to form the heterogeneous catalyst composition.

16. The process of claim 9, further comprising:
preparing the heterogeneous catalyst composition outside of the reactor system; and
loading the heterogeneous catalyst composition into the reactor system.

17. The process of claim 15, wherein the at least one metal catalyst precursor comprises a mixture of iron carbonate with lanthanum hydroxide, the boron precursor comprises boron oxide, and the matrix comprises a mixture of Li₂CO₃, Na₂CO₃, and K₂CO₃.

18. The process of claim 15, wherein preparing the heterogeneous catalyst composition outside of the reactor system comprises:
combining a mixture of alkali metal or alkaline earth metal salts or hydroxides to form a salt matrix comprising a eutectic mixture;
adding, to the matrix, a boron precursor and at least one metal catalyst precursor to form a catalyst precursor mixture; and
heating the catalyst precursor mixture to a temperature of from 390° C. to 750° C. to form the heterogeneous catalyst composition.

19. The process of claim 9, further comprising:
preparing the heterogeneous catalyst composition inside of the reactor system;
loading a catalyst precursor mixture into the reactor system;
wherein the catalyst precursor mixture comprises:
a matrix comprising a eutectic mixture of an alkali metal or alkaline earth metal salts or hydroxides, a boron precursor and at least one metal catalyst precursor selected from transition metal precursors, rare-earth metal precursors, alkaline-earth metal precursors, or a mixture thereof; and
heating at a temperature of about 390° C. to about 750° C.

20. The process of claim 18, wherein the at least one metal catalyst precursor comprises a mixture of iron carbonate with lanthanum hydroxide, the boron precursor comprises boron oxide, and the matrix comprises a mixture of Li₂CO₃, Na₂CO₃, and K₂CO₃.
